(19) United States
(12) Patent Application Publication
(10) Pub. No.: US 2023/0390517 A1
(43) Pub. Date: Dec. 7, 2023

(54) VENTILATION ACCESSORY DEVICE AND RELATED METHOD

(71) Applicants: Mahmoud Labib, Boston, MA (US); Mathew Peter Silvaggio, Bowmanville (CA)

(72) Inventors: Mahmoud Labib, Boston, MA (US); Mathew Peter Silvaggio, Bowmanville (CA)

(21) Appl. No.: 18/080,878

(22) Filed: Dec. 14, 2022

Related U.S. Application Data
(60) Provisional application No. 63/310,252, filed on Feb. 15, 2022.
(60) Provisional application No. 63/289,310, filed on Dec. 14, 2021.

(51) Int. Cl. A61M 16/08; A61M 16/06; A61M 16/10

(52) U.S. Cl. CPC A61M 16/0875; A61M 16/0605; A61M 16/1005

(57) ABSTRACT

In an artificial ventilation system, which includes a source of ventilation gas for delivery to the patient; a supply tube for conveying the gas; and a mask comprising an oronasal cup configured to form a cavity around a mouth and nose of a patient and an inlet in the cup and operatively supported at a downstream end of the supply tube distal to the source; there is provided an oral delivery conduit in the form of a tubular body configured to be received in the cavity and defining a path for flow of the ventilation gas. The tubular body is in fluidic communication with the supply tube and extends therefrom into the cavity and to a free end of the body arranged in proximal relation to the patient's mouth to convey the gas from the supply tube and towards the patient's mouth. An end portion of the tubular body defining the free end is arranged for sealingly gripping by the patient's lips for oral delivery of the ventilation gas, such that the mask forms a secondary seal for containing the gas relative to the patient's face.

This application claims the benefit under 35 U.S.C. 119(e) of U.S. provisional application Ser. No. 63/289,310 filed Dec. 14, 2021 and of U.S. provisional application Ser. No. 63/310,252 filed Feb. 15, 2022.

### FIELD OF THE INVENTION

The present disclosure relates generally to the process of ventilation and oxygenation of a patient and more specifically to a device and method for use in conjunction with a ventilation and/or oxygenation circuit to improve the efficacy of the ventilation and/or oxygenation process.

### BACKGROUND

In medicine, ventilation is the assistance of breathing by artificial or mechanical means. Generally, ventilation requires a source of gases that typically contains a mixture of room air and oxygen among other possible additives. The gas sources are typically titrated to varying levels and delivered through a circuit or conduit to a patient. The circuit or conduit is terminally attached to one of two devices that is applied to a patient to deliver the gas mixture; (1) a respiratory mask applied directly to the patient's face that typically covers their nose and mouth in an encapsulated covering or (2) an endotracheal tube or laryngeal mask placed above or into a patient's trachea by a process known as intubation. Ventilation delivered through a mask is typically referred to as non-invasive ventilation. Non-invasive ventilation can be delivered through various modes including assisted spontaneous ventilation, continuous positive airway pressure, and bi-level positive airway pressure among others. Ventilation delivered through an endotracheal tube or laryngeal mask is typically referred to as invasive ventilation and generally requires mechanical modes of controlled or assisted ventilation delivered through a ventilator.

Regarding the mixture of gases delivered through a conduit or circuit to a patient, when the mixture contains an oxygen level greater than that of room air, this type of ventilation is typically referred to as ‘oxygenation’. The intention of oxygenation is to provide a greater level of oxygen to a patient's lungs whereby increasing the amount of oxygen available for gas exchange to occur within their lungs. This is useful for many reasons including but not limited to, providing more oxygen to a patient who has increased oxygen requirements, providing increased oxygen available for gas exchange in a patient with lungs having a reduced capacity for gas exchange because of underlying pathology, or increasing the amount of oxygen available for a patient during a period of apnea.

Respiratory masks are used for ventilation and oxygenation in non-invasive forms. Respiratory masks generally have an opening for attachment of the gas circuit or conduit and is applied to a patient's face. The mask covering on a patient's face typically includes their nose and/or mouth. Effective ventilation and/or oxygenation to a patient is reliant on the integrity of the seal between the respiratory mask and patient's face. Respiratory masks have many limitations and shortcoming that can lead to an incomplete mask seal. This incomplete mask seal can lead to ineffective ventilation and/or oxygenation whereby a fraction of the delivered gas mixture is lost outside the mask, or room air is entrained into the mask. Additionally, aerosols generated within a patient's respiratory tract during ventilation and/or oxygenation can leak out of the mask and into the surrounding atmosphere.

Intubation is the process of inserting an endotracheal breathing tube into the trachea of an apneic patient. Completing the intubation within a short window of time is critical to avoiding long- and short-term damage which may result from a prolonged lack of oxygen going to the patient's organs and tissues. To reduce the risk of prolonged hypoxia and lengthen the window of time a medical practitioner has to correctly administer the breathing tube, the apneic patient is oxygenated prior to being intubated. Oxygenation involves administering up to 100% oxygen gas to the patient to increase the oxygen reserve in the patient's lungs and extend the period between the cessation of respiration of the patient and the onset of hypoxia (i.e., extending the period of tolerated apnea).

### SUMMARY OF THE INVENTION

According to an aspect of the present invention, there is provided a ventilation and/or oxygenation accessory device, comprising: an outer tube; and an inner tube disposed within the outer tube.

According to another aspect of the present invention, there is provided a method of improving the efficacy of a ventilation and/or oxygenation procedure, comprising: inserting a first end of an outer tube into an end of a length of tubing connected to a gas source; inserting a second end of the outer tube into an aperture in a respiratory mask; applying the respiratory mask to the face of a patient requiring ventilation and/or oxygenation; extending an inner tube, disposed within the outer tube, through the aperture in the respiratory mask such that the inner tube is proximate to the patient's mouth; and instructing the patient to wrap their lips around an end of the inner tube.

This effectively acts as a retractable conduit bridging the gap from the aperture of the respiratory mask to the patient's lips. When the patient's lips are sealed around the inner tube the integrity of ventilation and/or oxygenation is no longer reliant on the seal between the patient's face and respiratory mask but on the seal between the inner tube and patient's lips. This will improve the quality of ventilation and/or oxygenation by overcoming the inherent limitations of the respiratory mask. In the event that the patient is unable to complete a seal between their lips and the inner tube of the arrangement, quality of ventilation is still improved as the inner tube acts as a conduit to bridge the gap between the aperture of the respiratory mask and the patient's mouth. As the inner tube is retractable, in the event that a traditional mask seal is required, retraction of the inner tube into the outer tube re-establishes the traditional mask seal.

According to another aspect of the invention, there is provided a method of improving the efficacy of ventilation and/or oxygenation procedure, comprising: inserting a first end of an outer tube into an end of a length of tubing connected to an oxygen or gas source; inserting a first end of an inner tube into a second end of the outer tube; inserting the second end of the outer tube into an aperture in a respiratory mask; applying the respiratory mask to the face of a patient requiring ventilation and/or oxygenation; and instructing the patient to wrap their lips around an end of the inner tube.

This effectively acts as a conduit bridging the gap from the aperture of the respiratory mask to the patient's lips. When the patient's lips are sealed around the inner tube the integrity of ventilation and/or oxygenation is no longer reliant on the seal between the patient's face and respiratory mask but on the seal between the inner tube and patient's lips. This will improve the quality of ventilation and/or oxygenation by overcoming the inherent limitations of the respiratory mask. In the event that the patient is unable to complete a seal between their lips and the inner tube of the arrangement, quality of ventilation is still improved as the inner tube acts as a conduit to bridge the gap between the aperture of the respiratory mask and the patient's mouth.

According to another aspect of the invention there is provided an oxygenation circuit accessory device comprising an outer tube and an inner tube disposed within the outer tube.

In one arrangement, the device further comprise an inner tube retraction and extension mechanism.

In one arrangement, the device further comprises a respiratory mask.

In one arrangement, a first end of the outer tube is configured to connect to an aperture in a respiratory mask.

In one arrangement, a second end of the outer tube is configured to connect to a length of tubing, the length of tubing being further connected to an oxygen source.

In one arrangement, the inner tube is of a length lesser than or equal to a length of the outer tube.

In one arrangement, the inner tube is of a length greater than or equal to a length of the outer tube.

In one arrangement, an end of the inner tube comprises a mouthpiece.

In one arrangement, at least one end of the outer tube is tapered.

In one arrangement, the inner tube retraction and extension mechanism comprises:
a grooved track embedded within an outer face of the inner tube and parallel to a length of the inner tube;
an aperture in the outer tube; and
a toothed wheel mounted in the aperture, immediately above the grooved track, its rotational axis perpendicular to a length of the outer tube.

In one arrangement, the inner tube retraction and extension mechanism comprises:
an elongated primary channel in a wall of the outer tube, wherein the primary channel is parallel to a length of the outer tube; and
a lever extending from a wall of the inner tube and through the primary channel.

In one such arrangement, the primary channel comprises at least one side channel, the at least one side channel extending from an end of the primary channel.

In one arrangement, the inner tube retraction and extension mechanism comprises:
a retractable compressive element disposed between the inner tube and the outer tube, wherein the compressive element, in an extended position, creates a kink in the inner tube.

In one arrangement, the inner tube retraction and extension mechanism comprises a latch mechanism similar to that which may be employed in a retractable pen.

According to another aspect of the invention there is provided a method of improving the efficacy of an oxygenation procedure, comprising:
inserting a first end of an outer tube into an end of a length of tubing connected to an oxygen source;
inserting a second end of the outer tube into an aperture in a respiratory mask; applying the respiratory mask to the face of a patient requiring oxygenation;
extending an inner tube, disposed within the outer tube, through the aperture in the respiratory mask such that the inner tube is proximate the patient's mouth; and
instructing the patient to wrap their lips around an end of the inner tube.

In one arrangement, the step of extending the inner tube comprises rotating a toothed wheel mounted within an aperture in a wall of the outer tube, such that the toothed wheel engages a length of grooved track embedded in an outer face of the inner tube and running parallel to a length thereof.

In one arrangement, the step of extending the inner tube comprises:
gripping a lever extending from a wall of the inner tube and through a primary channel in a wall of the outer tube; and
sliding the lever toward an end of the primary channel.

In one arrangement, the step of extending the inner tube comprises retracting a compressive element disposed between the inner tube and the outer tube.

According to another aspect of the invention there is provided method of improving the efficacy of an oxygenation procedure, comprising:
inserting a first end of an outer tube into an end of a length of tubing connected to an oxygen source;
inserting a first end of an inner tube into a second end of the outer tube;
inserting the second end of the outer tube into an aperture in a respiratory mask;
applying the respiratory mask to the face of a patient requiring oxygenation; and
instructing the patient to wrap their lips around a second end of the inner tube.

According to another aspect of the invention there is provided a method of improving the efficacy of an oxygenation procedure, comprising:
inserting a device through a mask, wherein one end of the device is connected to an oxygen source and the other end of the device is proximate a patient's mouth, and wherein the device is a single piece.

According to another aspect of the invention there is provided a device for use in a system to artificially ventilate a patient, wherein the system includes a source of ventilation gas for delivery to the patient, a supply tube configured to convey the ventilation gas and having an upstream end in fluidic communication with the source to receive the ventilation gas therefrom and a downstream end configured to release the gas, and a mask comprising an oronasal cup configured to form a cavity around a mouth and nose of the patient and an inlet in the oronasal cup and operatively supported at the downstream end of the supply tube, the device comprising:
a tubular body defining a path for flow of the ventilation gas;
wherein the tubular body is configured to be received in the cavity;
wherein the tubular body has a first end arranged in fluidic communication with the downstream end of the supply tube and a second end arranged in proximal relation to the mouth of the patient, so as to be arranged to extend from the downstream end of the supply tube and into the cavity towards the patient's mouth;
wherein an end portion of the tubular body defining the second end thereof is arranged for sealingly gripping by lips of the patient for oral delivery of the ventilation gas, such that the mask forms a secondary seal for containing the ventilation gas relative to a face of the patient.

According to another aspect of the invention there is provided a system for artificially ventilating a patient comprising:
a source of ventilation gas for delivery to the patient;
a supply tube configured to convey the ventilation gas and having an upstream end in fluidic communication with the source to receive the ventilation gas therefrom and a downstream end configured to release the gas;
a mask comprising an oronasal cup configured to form a cavity around a mouth and nose of the patient and an inlet in the oronasal cup and operatively supported at the downstream end of the supply tube;
an oral delivery conduit in the form of a tubular body configured to be received in the cavity and defining a path for flow of the ventilation gas;
wherein the tubular body has a first end in fluidic communication with the downstream end of the supply tube and extends therefrom into the cavity and to a second end arranged in proximal relation to the mouth of the patient, so as to convey the ventilation gas from the supply tube and towards the patient's mouth;
wherein an end portion of the tubular body defining the second end thereof is arranged for sealingly gripping by lips of the patient for oral delivery of the ventilation gas, such that the mask forms a secondary seal for containing the ventilation gas relative to a face of the patient.

Either of the two aforementioned aspects provide an arrangement of oral delivery conduit for bridging, within an oronasal cavity formed by the mask, a distance between an inlet of the oronasal cup, proximate to which the downstream end of the supply tube is located, and the patient's mouth, so that the ventilation gas is released at a position closer to the patient's mouth for inhaling by the patient using the mouth. Especially when the patient wraps their lips around the end portion of the conduit, a transfer of ventilation gas to a respiratory system of the patient, and particularly their lungs, is enhanced as compared to flooding the oronasal cavity with the ventilation gas because the patient's lips can substantially conform to an outer surface of the body of the conduit to form a relatively impermeable seal therebetween as compared to conformability of the mask to the patient's face for sealing purposes. In this manner, for a prescribed duration of time, a greater proportion of the supplied ventilation gas may be delivered to the patient's lungs, and accordingly a lesser proportion of the supplied ventilation gas may be lost to a surrounding environment.

In the illustrated arrangements, the end portion of the tubular body is cylindrical in shape.

Preferably, in a working condition in which the end portion is arranged at the mouth of the patient so as to be available for gripping by the lips of the patient, the tubular body extends linearly between the first and second ends thereof.

In some arrangements, the tubular body comprises an outer section defining the first end and an inner section defining the end portion and operatively interconnected to the outer section to provide a continuous path for flow of the ventilation gas, and wherein the outer section is arranged fluidically in series between the supply tube and the mask.

In some arrangements, when the oronasal cup has a rim configured for sealing engagement with the patient's face, the tubular body has adjustable length between the first and second ends so as to be configurable between a retracted position in which the second end is arranged closer to the inlet of the mask than to the rim of the oronasal cup and an extended position in which the second end is arranged closer to the rim of the oronasal cup than to the inlet.

In one such arrangement, when the tubular body comprises a first section defining the first end and a second section defining the end portion and configured for movement relative to the first section to provide the adjustable length, the first and second sections are operatively interconnected for relative movement by a mechanism comprising a track supported on one of the first and second sections and a wheel operatively supported on another one of the first and second sections and matably engaged with the track.

In another arrangement, when the tubular body comprises a first section defining the first end and a second section defining the end portion and configured for movement relative to the first section to provide the adjustable length, the second section is fixedly attached to the first section and comprises a flexible length of tubing deformable crosswise to a longitudinal direction of the tubular body for varying the adjustable length.

In another arrangement, the first section forms an outer housing having an interior through which the second section extends, and the tubular body includes a deflector supported in the outer housing and movable relative thereto in the crosswise direction for deformatively engaging the second section.

In another arrangement, when the tubular body comprises a first section defining the first end and a second section defining the end portion and configured for movement relative to the first section to provide the adjustable length, the first section forms an outer housing having an interior through which the second section extends, and the second section is fixedly attached to the first section and is compressible in a longitudinal direction of the tubular body for providing the adjustable length.

In another arrangement, when the tubular body comprises a first section defining the first end and a second section defining the end portion and configured for movement relative to the first section to provide the adjustable length, the first and second sections are telescopically slidably interconnected and are rotatable to each other around a longitudinal axis of the tubular body, and wherein an outer one of the first and second sections includes a slot extending longitudinally of the tubular body and an inner one of the first and second sections includes a catch received in the slot, wherein the slot includes jogs in crosswise directions to the longitudinal direction for maintaining the first and sections at a selected length.

In one arrangement, the tubular body comprises a first section defining the first end and a second section defining the end portion and distinct from the first section, and the first and second sections are removably interconnectable in fixed relation to each other.

According to another aspect of the invention there is provided a method for artificially ventilating a patient comprising:
providing a source of ventilation gas for delivery to the patient;
providing a supply tube operatively connected to the source to convey the ventilation gas therefrom and to a release location in proximal spaced relation to a face of the patient and defined by an end of the supply tube distal to the source;
providing a mask in operative association with the supply tube to form at the release location a cavity around a mouth and nose of the patient for containing the ventilation gas relative to a face of the patient;
providing a tubular conduit distinct from the supply tube and fluidically communicated with the distal end thereof to convey the ventilation gas from the supply tube and towards the mouth of the patient;
locating the mask on the patient's face to form the cavity; and
locating the tubular conduit in the cavity to present an end portion thereof, which defines a free end of the tubular conduit, for gripping by lips of the patient for oral delivery of the ventilation gas.

In some arrangements, locating the tubular conduit in the cavity comprises extending the end portion towards the mouth of the patient from a retracted position in which the free end is in outwardly spaced relation from the mouth of the patient such that the end portion is unavailable for gripping by the lips of the patient.

The method may further include, after locating the tubular conduit in the cavity, substantially removing the tubular conduit from the cavity and maintaining the mask on the patient's face so that the mask acts to convey the ventilation gas for oronasal delivery to the patient.

In one such arrangement, substantially removing the tubular conduit from the cavity comprises retracting the end portion away from the mouth of the patient from an extended position in which the end portion is at the mouth of the patient to be available for gripping by the lips of the patient.

Preferably, retracting the end portion is performed while the mask is on the patient's face forming the cavity.

Other aspects and features according to the present application will become apparent to those ordinarily skilled in the art upon review of the following description of arrangements or embodiments of the invention in conjunction with the accompanying figures.

### BRIEF DESCRIPTION OF THE DRAWINGS

The principles of the invention may better be understood with reference to the accompanying figures provided by way of illustration of an exemplary arrangement(s) or embodiment(s), incorporating principles and aspects of the present invention, and in which:

FIG. 1 is a schematic diagram of a conventional circuit or system for artificially ventilating or oxygenating a patient;

FIG. 2 is a schematic diagram of an arrangement of system for artificially ventilating a patient according to the present invention;

FIGS. 3A and 3B are enlarged partial views of the system of FIG. 1 showing a first arrangement of oral delivery conduit in extended and retracted positions, respectively;

FIGS. 4 through 8, respectively, are perspective, top plan, side and cross-sectional views of a second arrangement of oral delivery conduit shown in a retracted condition, where the cross-section in FIG. 7 is taken along line 7-7 in FIG. 5 and the cross-section in FIG. 8 is taken along line 8-8 in FIG. 5;

FIGS. 9 through 12, respectively, are perspective, top plan, side and cross-sectional views of the third arrangement of oral delivery conduit shown in an extended condition, where the cross-section in FIG. 12 is taken along line 12-12 in FIG. 10;

FIGS. 13 and 14 are top plan and cross-sectional views, respectively, of a third arrangement of oral delivery conduit shown in a retracted condition, where the cross-section in FIG. 14 is taken along line 14-14 in FIG. 13;

FIGS. 15 and 16 are top plan and cross-sectional views, respectively, of the third arrangement of oral delivery conduit shown in an extended condition, where the cross-section in FIG. 16 is taken along line 16-16 in FIG. 15;

FIG. 17 is a side view of a fourth arrangement of oral delivery conduit shown in a retracted condition;

FIGS. 18 and 19 are plan and cross-sectional views, respectively, of the fourth arrangement in the retracted condition, where the cross-section in FIG. 19 is taken along line 19-19 in FIG. 18;

FIGS. 20 and 21 are plan and cross-sectional views, respectively, of the fourth arrangement shown in an extended condition, where the cross-section in FIG. 21 is taken along line 21-21 in FIG. 20;

FIG. 22 is a side view of a fifth arrangement of oral delivery conduit shown in a retracted condition;

FIGS. 23 and 24 are plan and cross-sectional views, respectively, of the fifth arrangement shown in an extended condition, where the cross-section in FIG. 24 is taken along line 24-24 in FIG. 23;

FIGS. 25 and 26 are plan and cross-sectional views, respectively, of the fifth arrangement in the retracted condition, where the cross-section in FIG. 26 is taken along line 26-26 in FIG. 25;

FIG. 27 is a perspective view of a sixth arrangement of oral delivery conduit;

FIG. 28 is an elevational view of the sixth arrangement of FIG. 27;

FIG. 29 is a cross-sectional view along line 29-29 in FIG. 28;

FIG. 30 is an enlarged partial view of the area indicated at I in FIG. 29; and

FIGS. 31 and 32 are exploded views of the sixth arrangement of FIG. 27.

In the drawings like characters of reference indicate corresponding parts in the different figures.

### DETAILED DESCRIPTION

The description that follows, and the arrangements described therein, are provided by way of illustration of an example, or examples, of particular arrangements of the principles of the present invention. These examples are provided for the purposes of explanation, and not of limitation, of those principles and of the invention. In the description, like parts are marked throughout the specification and the drawings with the same respective reference numerals. The drawings are not necessarily to scale and, in some instances, proportions may have been exaggerated in order more clearly to depict certain features of the invention.

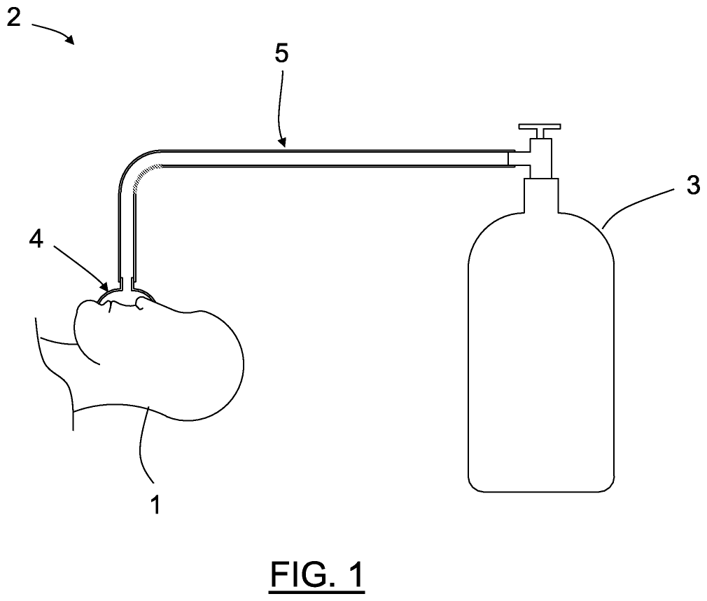

With reference to FIG. 1, oxygenation, or more generally artificial ventilation, of a patient 1 is achieved through the use of a circuit or system 2 comprising a source of gas 3, for example oxygen, connected to a respiratory mask 4, most often by a length of tubing 5. The efficacy of the process is largely dependent on the quantity or fraction of the oxygen from the oxygen source which enters the patient's body and, more specifically, lungs. This quantity will be reduced when, as is commonly the case, there is an incomplete seal between the respiratory mask, held in place by a medical practitioner, and the patient's face. The integrity of the seal may be compromised due to human error (i.e., the mask being incorrectly applied by the medical practitioner), but a less-than-complete seal may also result from differences in patients' facial shapes and structures. Respiratory masks are generally created as one-size-fits-all and therefore may be challenging to use effectively on patients with widely variable facial features. For example, it may be difficult if not impossible to achieve a complete seal on a patient with a larger nose or narrower face, a patient with a significant amount of facial hair, or a patient with a history of surgical resection of the head or neck.

Not only can an incomplete seal between the patient's face and the mask compromise the efficacy of the critical oxygenation process, and thereby increase the risk of organ or tissue damage due to hypoxia, it may also expose medical professionals to respiratory pathogens emanating from the patient's airways and escaping through gaps between the mask and the patient's face.

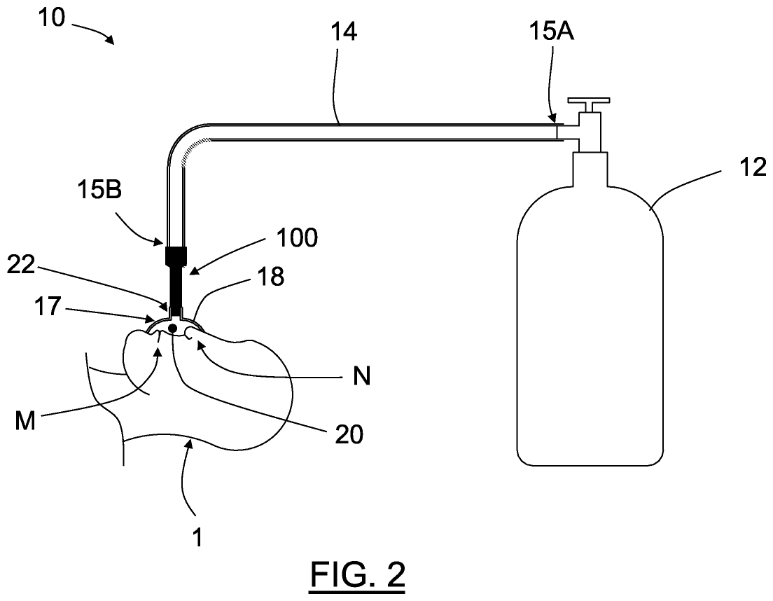

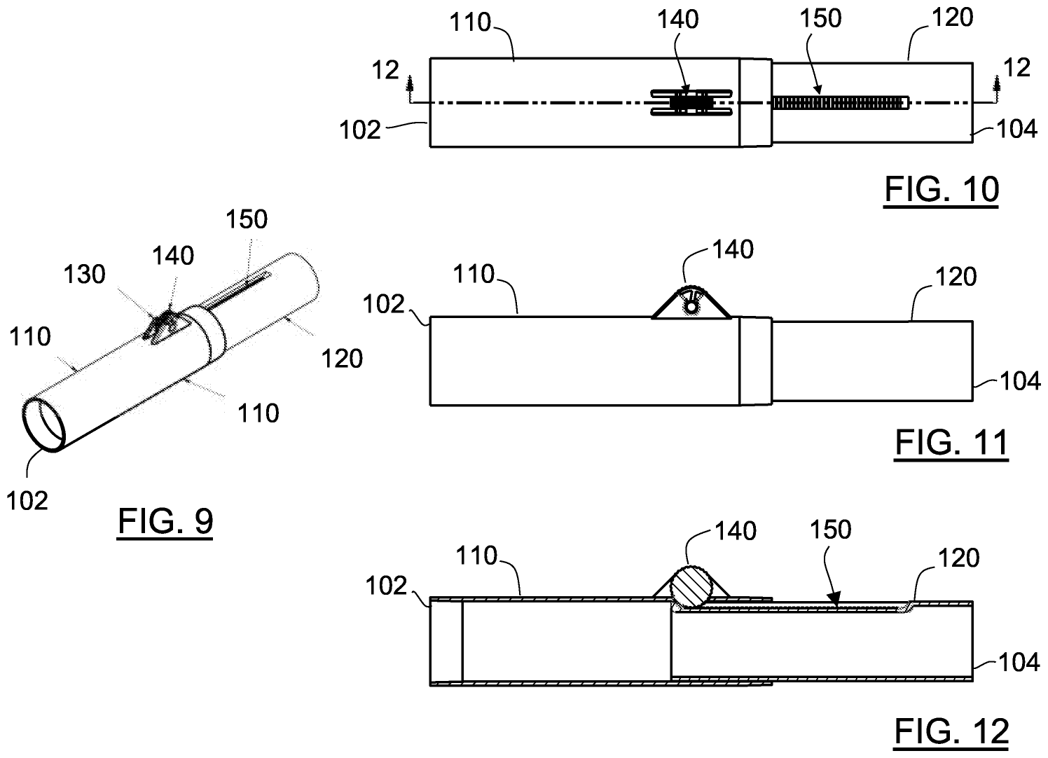

Thus is shown in FIGS. 2 through 32 a system 10 for artificially ventilating a patient including an oral delivery conduit 100 for guiding ventilation gas to a release location in close relation to the patient's mouth. Referring to FIG. 2, the system 10 generally comprises:
a source of ventilation gas 12 for delivery to the patient 1;
a supply tube 14 configured to convey the ventilation gas and having an upstream end 15A in fluidic communication with the source 12 to receive the ventilation gas therefrom and a downstream end 15B configured to release the gas;
a mask 17 comprising an oronasal cup 18 configured to form a cavity 20 around a mouth M and nose N of the patient 1 and an inlet 22 in the oronasal cup and operatively supported at the downstream end 15B of the supply tube; and
the oral delivery conduit 100 in the form of a tubular body configured to be received in the cavity 20 and defining a path for flow of the ventilation gas.

The mask's oronasal cup 18 acts to form the cavity 20 in a working condition of the mask in which a rim 25 of the cup 18, that is a terminal peripheral edge delimiting an open end of the mask in opposite relation to the inlet and configured to receive an oronasal portion of the patient's face, and furthermore configured for sealing engagement with the patient's face F, is located or placed in substantially sealing engagement with the patient's face and in a manner surrounding the mouth and nose of the patient. The oronasal portion of the face includes the nose, and particularly an end portion thereof proximal nostrils of the nose; the mouth; epidermal tissue between the nose and an upper one of the lips of the mouth; and epidermal tissue below a lower one of the lips of the mouth. Typically, the cup 18 is generally dome-shaped, with the inlet 22 being located closer to an apex of the dome-shaped cup than to the rim 25.

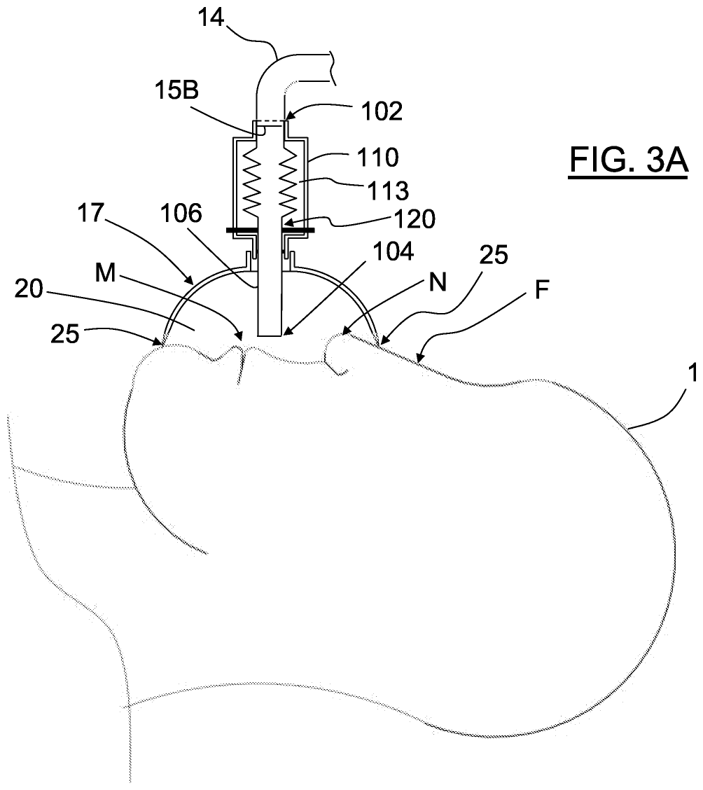

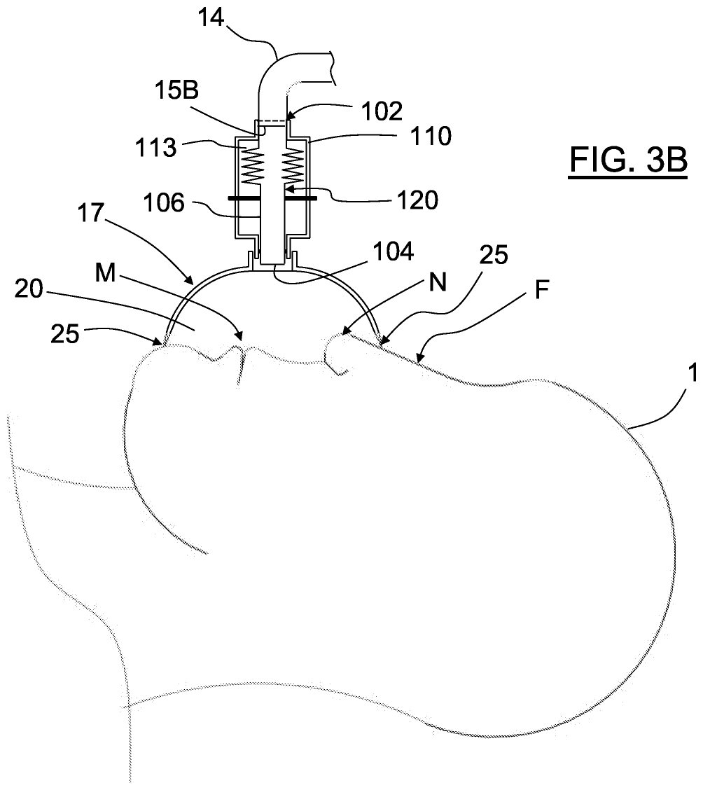

As more clearly shown in FIGS. 3A and 3B, the tubular conduit body 100 has a first end 102 in fluidic communication with the downstream end 15B of the supply tube 14 and extends therefrom into the cavity 20 and to a second end 104 arranged in proximal relation to the mouth M of the patient, so as to convey the ventilation gas from the supply tube and towards the patient's mouth. That is, the ventilation gas guided by the supply tube 14 is passed or transferred to the oral delivery conduit 100, and not to the cavity 20 for example via the inlet 22 of the mask, for release substantially at the mouth.

An end portion 106 of the tubular body defining the second end 104 thereof is arranged for sealingly gripping by lips of the patient for oral delivery of the ventilation gas, such that the mask 17 forms a secondary or auxiliary seal for containing the ventilation gas relative to the face F of the patient. That is, when the patient's lips are gripping the end portion 106 in such a manner as to substantially form a seal between an outer surface of the end portion and the patient's lips, so that substantially all of the ventilation gas released from the conduit is transferred to the patient's mouth and more generally a respiratory system thereof, a primary seal is formed between the patient's lips and the end portion 106 of the tubular body, and any gas which may escape the end portion and/or the patient's respiratory system, for example by the nose, during transfer of the ventilation gas to the patient is contained within the cavity by the seal formed between the mask 17 and the patient's face F.

The oral delivery conduit may be alternatively referred to as a ventilation and oxygenation accessory device 100, which is provided to improve the efficacy of the ventilation and/or oxygenation process. Accordingly, ventilation and oxygenation accessory device 100 may reduce the risk of onset of hypoxia in the time between a patient is rendered apneic and the successful intubation of a patient and increase the amount of time available to a medical practitioner to successfully intubate. By reducing the risk of hypoxia, ventilation and oxygenation accessory device 100 may also reduce the risk of associated hypoxic organ and tissue injury. Further, ventilation and oxygenation accessory device 100 may reduce the physical strain which may be experienced by medical practitioners while attempting to manually create a complete seal between a respiratory mask of an oxygenation circuit and a patient's face.

Ventilation and oxygenation accessory device 100 may be incorporated into a ventilation or oxygenation circuit between a respiratory mask and a length of tubing connected to a ventilation gas source, for example oxygen.

In an extended configuration, an inner tube of ventilation and oxygenation accessory device 100 extends into the respiratory mask and can be engaged by a conscious patient. The patient may engage the inner tube by wrapping their lips around the inner tube. Oxygen flowing from the oxygen source through the length of tubing and outer tube then flows through the inner tube directly into the body of the patient and is not lost at the site of the respiratory mask through gaps between the respiratory mask and the patient's face. In the event that the patient is unconscious or uncooperative, the inner tube may be retracted such that traditional oxygen delivery into a cavity defined by the respiratory mask and the patient's face is possible without first disconnecting the ventilation and oxygenation accessory device 100 from the oxygenation circuit. The inner tube may also be quickly retracted and traditional oxygen delivery resumed in the event that a patient becomes unconscious or uncooperative during oxygenation. According to an arrangement, ventilation and oxygenation accessory device 100 is incorporated into an oxygenation circuit as a terminal component at an end of a length of tubing connected to an oxygen source proximate a patient. It will be appreciated that the accessory device 100 is considered generally coterminous with the respiratory mask in the circuit, such that when substantially retracted from the cavity, the ventilation gas fills or floods the oronasal cavity 20 formed by the mask around the mouth and nose of the patient.

In other words, in some arrangements the tubular body 100 comprises a first outer section 110 defining the first end 102 and a second inner section 120 defining the end portion 106, and accordingly the second end 104, and operatively interconnected to the first section 110 to provide a continuous path for flow of the ventilation gas. In the illustrated arrangements, and more clearly shown in FIGS. 3A and 3B, the outer section 110 is arranged fluidically in series between the supply tube 14 and the mask 17. In such an arrangement, the oral delivery conduit acts to operatively support the inlet 22 of the mask at the downstream end of the supply tube, such that the ventilation gas passes from the supply tube and through the inlet but is not guided by the cup 18, rather by the tubular conduit 100.

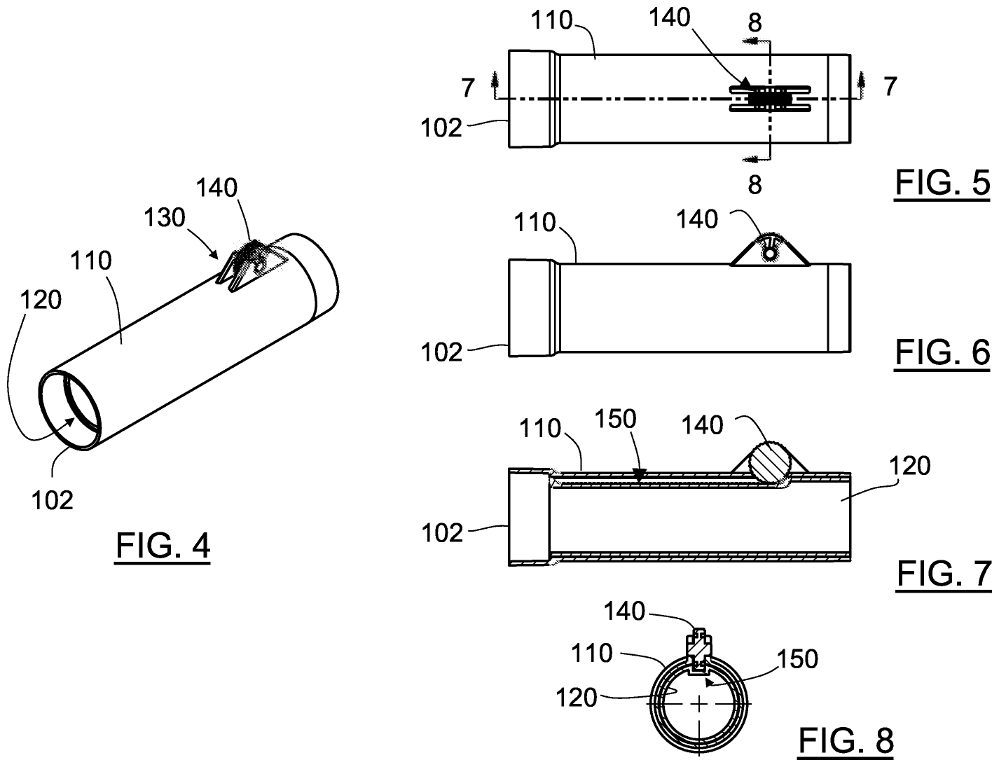

In the illustrated arrangements, the ventilation and oxygenation accessory device 100 generally includes an outer tube 110 and an inner tube 120, disposed within outer tube 110. In one of the arrangements, outer tube 110 has a diameter of 22 mm. Outer tube 110 may be 85 mm long. According to an arrangement, a first end of outer tube 110 is configured to connect to an aperture in a respiratory mask. A second end of outer tube 110 may be configured to connect to a length of tubing, the length of tubing being further connected to an oxygen or gas source. At least one end of outer tube 110 may be tapered. At least one end of outer tube 110 may have a 1:40 taper. Outer tube 110 having a taper may facilitate the connection of outer tube 120 at a first end with an aperture in a respiratory mask and at a second end with a length of tubing making up a ventilation and/or oxygenation circuit. At least one end of outer tube 110 may have an 8 mm taper length. Inner tube 120 may have a diameter of 19.75 mm. Inner tube 120 may be 75 mm long. Inner tube 120 may be of a length lesser than or equal to a length of outer tube 110. Alternatively, inner tube 120 may be of a length greater than or equal to a length of outer tube 110. Inner tube 120 may, in an extended position, extend no more than 50 mm past an end of outer tube 110, as shown in FIGS. 4 through 12. An end of inner tube 120 may comprise a mouthpiece. A mouthpiece disposed on an end of inner tube 120 may reduce an amount of effort for a patient to create a lip seal around an end of inner tube 120 when ventilation and oxygenation accessory device 100 is used with a ventilation and/or oxygenation circuit.

When the oral delivery conduit comprises first and second tubular sections, the ventilation and oxygenation accessory device 100 may include an inner tube retraction and extension mechanism 130. Inner tube retraction and extension mechanism 130 may permit use of ventilation and oxygenation accessory device 100 with a ventilation and/or oxygenation circuit irrespective of a state of consciousness of a patient to which the ventilation oxygenation circuit is applied. When the patient is conscious and cooperative, inner tube retraction and extension mechanism 130 may be used to extend inner tube 120 into a respiratory mask applied to the patient's face, enabling the patient to engage inner tube 120 with their lips. In the event of an uncooperative or unconscious patient, unable or unwilling to engage inner tube 120 in this manner, inner tube retraction and extension mechanism 130 may be used to retract inner tube 120 into outer tube 110 such that oxygen is delivered more traditionally into the entire respiratory mask.

In other words, the tubular body 100 has adjustable length between the first and second ends 102, 104 so as to be configurable between a retracted position in which the second end 104 is arranged closer to the inlet 22 of the mask 17 than to the rim 25 of the oronasal cup 18, as more clearly shown in FIG. 3B, and an extended position in which the second end 104 is arranged closer to the rim 25 of the oronasal cup than to the mask inlet 22. In the extended position, the end portion 106 is substantially inside the cavity 20, that is a majority portion or length of the end portion 106 is disposed inside the cavity 20. In contrast, in the retracted position, the end portion 106 is substantially external to the cavity and a majority portion or length thereof is located upstream to the inlet 22, although the second end 106 may be inside the cavity 20. Configurability between the retracted and extended positions relative to the mask may be provided by movement of distinct tubular sections forming the conduit 100, as in the illustrated arrangements.

More specifically, the tubular body 100 comprises a first section 110 defining the first end 102 and a second section 120 defining the end portion 106 and configured for movement relative to the first section to provide the adjustable length.

In one arrangement of delivery conduit with adjustable length as shown in FIGS. 3A and 3B, the first section 110 forms an outer housing having an interior 113 through which the second section 120 extends, and the second section 120 is fixedly attached to the first section 110 and is compressible in a longitudinal direction of the tubular body for providing the adjustable length. The longitudinal direction is directed from the first end 102 to the second end 104 and also corresponds to a direction of flow of ventilation gas through the conduit in the ventilation system.

According to an arrangement, as shown in FIGS. 4-12, inner tube retraction and extension mechanism 130 comprises: a grooved track 150 embedded within an outer face of inner tube 120 and parallel to a length of inner tube 120; an aperture in outer tube 110; and a toothed wheel 140 mounted in the aperture, immediately above grooved track 150, its rotational axis perpendicular to a length of outer tube 110. Toothed wheel 140 may be spun in a first direction to retract inner tube 120 and in a second direction to extend inner tube 120. Spinning toothed wheel 140 causes toothed wheel 140 to push inner tube 120 forward or backward by engaging a plurality of grooves in grooved track 150.

In other words, the first and second sections 110, 120 are operatively interconnected for relative movement by a mechanism comprising a track 150 supported on one of the first and second sections, in this case an inner one of the sections 120, and a wheel 140 operatively supported on another one of the first and second sections, in this case an outer one of the sections 110, and which wheel is matably engaged with the track 150. Thus, rotation of the wheel acts to displace the section of the conduit supporting the track 150 relative to the section of the conduit rotatably supporting the wheel 140. In this arrangement, and as shown in more clearly in FIG. 7, the outer and inner sections may be telescopically configured, such that the inner section 120 retracts into the outer section, and furthermore the inner section 120 may be configured to be received wholly within the outer section 110 in a retracted condition of the conduit, which condition corresponds to the retracted position of the conduit relative to the mask when assembled in the system 10.

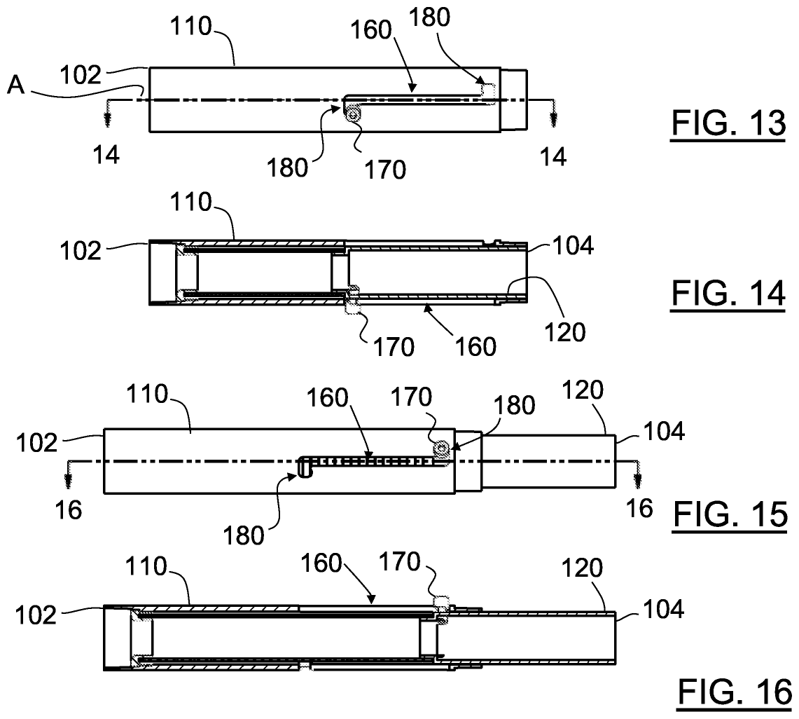

According to another arrangement, as those shown in FIGS. 13-21, inner tube retraction and extension mechanism 130 comprises an elongated primary channel 160 in a wall of outer tube 110, wherein primary channel 160 is parallel to a length of outer tube 100; and a lever 170 extending from a wall of inner tube 120 and through primary channel 160. Lever 170, being in connection with inner tube 120, may be used to extend inner tube 120 by sliding lever 170 along primary channel 160 in a first direction and to retract inner tube 120 by sliding lever 170 along primary channel 160 in a second direction opposite the first direction. In the aforementioned arrangements, primary channel 160 comprises at least one side channel 180, at least one side channel 180 extending from an end of primary channel 160. At least one side channel 180 may permit the position of inner tube 120 to be secured by directing lever 170 to an end of primary channel 160 and into at least one side channel 180. By removing lever 170 from elongated primary channel 160, the inadvertent retraction or extension of inner tube 120 resulting from the repositioning of lever 170 in primary channel 160 may be reduced. At least one side channel 180 may therefore serve to lock the position of inner tube 120.

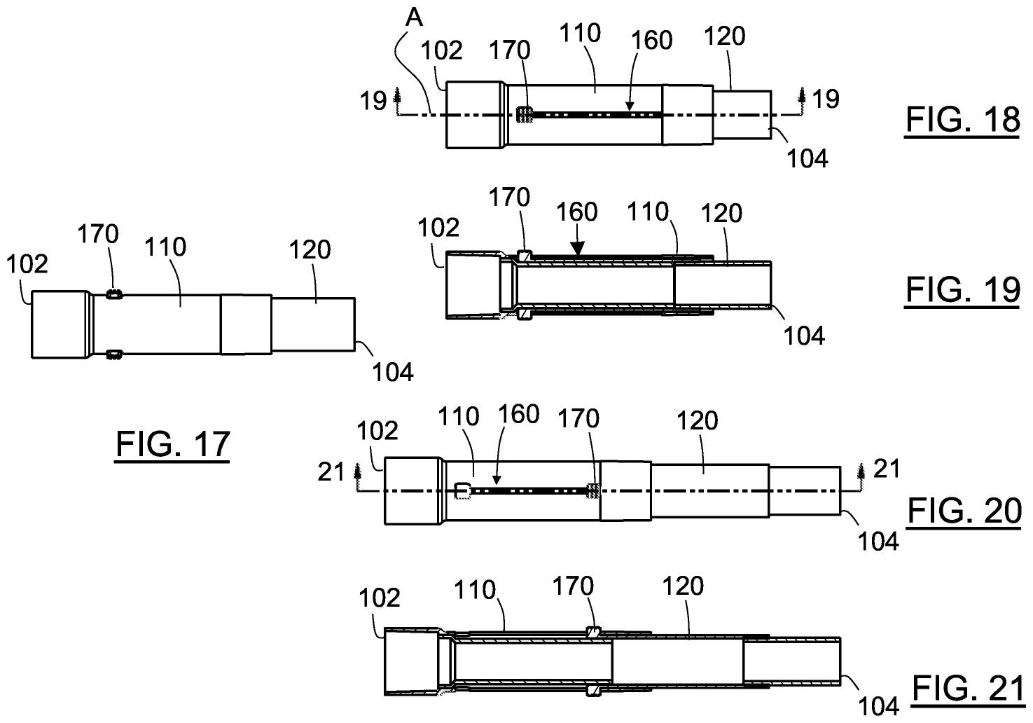

In other words, in these arrangement the first and second sections 110, 120 are telescopically slidably interconnected and an outer one of the sections, in this case 110, includes a slot 160 extending longitudinally of the tubular body and an inner one of the sections, in this case 120, includes a catch 170 received in the slot. The slot 160 includes jogs 180 in crosswise directions to the longitudinal direction for maintaining the first and sections at a selected length. In one arrangement such as that shown in FIGS. 13-16, the first and second sections 110, 120 are rotatable to each other around a longitudinal axis A of the tubular body and the crosswise direction in which the jogs extend from the slot may be circumferential or angular relative to the axis A. In another arrangement such as that shown in FIGS. 17-21, the crosswise direction may be a radial direction relative to the axis A of the tubular body, and in such an arrangement the first and second sections may be fixed in rotational relation to each other.

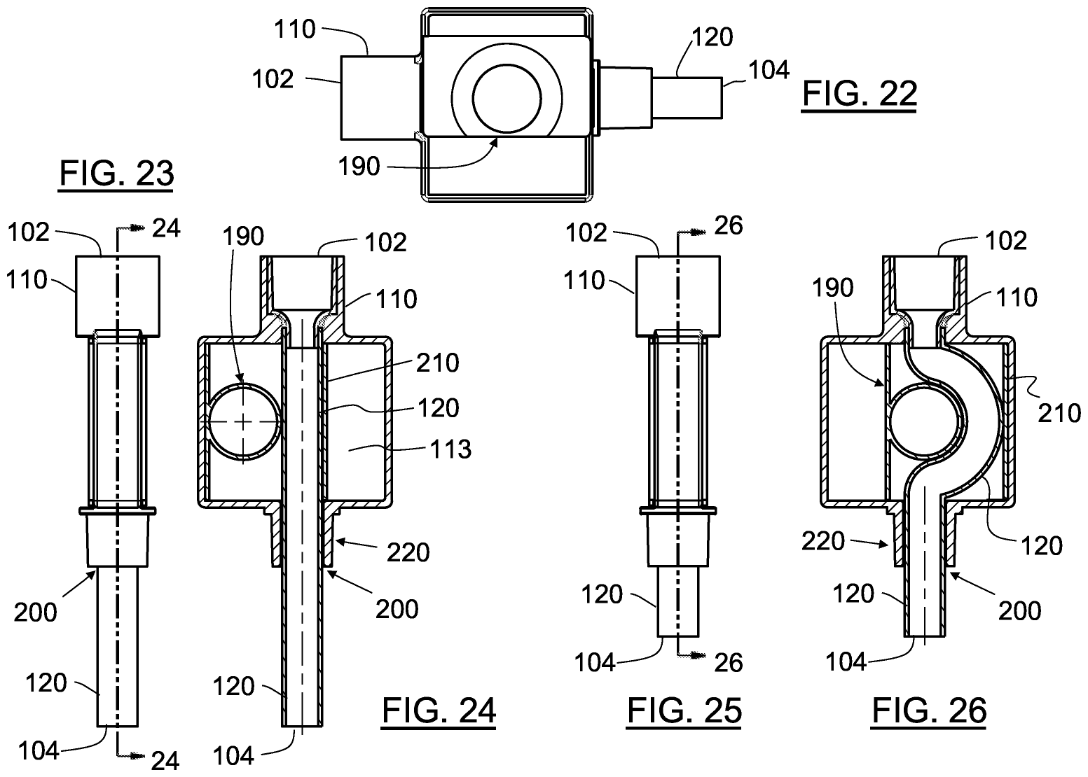

In another arrangement, as shown in FIGS. 22-26, inner tube retraction and extension mechanism 130 comprises a retractable compressive element 190 disposed between inner tube 120 and outer tube 110, wherein compressive element 190, in an extended position, creates a kink in inner tube 120. The kink in inner tube 120 shortens a linear distance between a first end of inner tube 120, closest to a respiratory mask of an oxygenation circuit to which ventilation and oxygenation accessory device 100 is applied, and a second end of inner tube 120. Compressive element 190 being in the extended position therefore causes inner tube 120 to retract into outer tube 110. With compressive element 190 in a retracted position, pressure exerted on inner tube 120 by compressive element 190 is relieved, the kink in inner tube 120 is thereby eliminated, and a length of inner tube 120 which extends past a first end of outer tube 110 increases. Alternatively, inner tube retraction and extension mechanism 130 may comprise a latch mechanism similar to that which may be employed in a retractable pen.

In other words, in this arrangement, the second section defining the end portion 106, in this case the inner section 120, is fixedly attached to the first section 110 and comprises a flexible length of tubing deformable crosswise to a longitudinal direction of the tubular body for varying the adjustable length. In such an arrangement, the first section 110 forms an outer housing having an interior 113 through which the second section 120 extends, and the tubular body includes a deflector 190 supported in the outer housing, that is in the interior 113 thereof, and movable relative to the housing in the crosswise direction for deformatively engaging the second section 120. More specifically, the flexible second section 120 is resiliently deformable so as to be repositionable between an extended condition relative to the housing, in which an external portion 190 of the second section 120, which is disposed outside the housing 110, has a maximum length between a downstream end 200 of the housing, through which the second section 120 passes, and a free terminus of the second section defined by the second end 104; and a retracted condition relative to the housing, in which the external portion 190 has minimum length between the downstream housing end 200 and the free end 104. In the illustrated arrangement, when in the extended condition, the flexible tubing follows a linear path, so that an internal portion of the second section located inside the housing traverses a shortest distance between a mounting location in the housing, to which a fixed end of the second section is attached, and the downstream end 200. In the retracted condition, the internal portion of the flexible second section follows a curved path so as to traverse a longest distance between the mounting location and the downstream end of the housing 200. Furthermore, it will be appreciated that there is provided a stopper 210 in the form of a rigid planar member carried by the flexible second section 120 in opposite relation to the deflector 190 and in fixed relation to the flexible section 120 for butting engagement with an interior wall of the housing to delimit a maximum deflection of the second section, which is associated with the retracted condition. In the illustrated arrangement, the deflector 190 and the stopper 210 are respectively slidably supported within the housing formed by the first section 110. Moreover, the deflector 190 has a rounded contact surface for deformatively bending the second section to form a curved path. A tubular sleeve or collar 220 is provided at the downstream end 200 of the housing to support the external portion of the second section to extend linearly, that is along a linear path, to the free end 104.

According to another arrangement, a device 100 includes a single piece. One end of the device is connectible to a ventilation gas source (e.g., which can be connectible to the mask) and the other end of the device 100 is insertable through an opening in the mask and can be placed proximate a patient's mouth. The device 100 does not include separate movable pieces that extend or retract relative to each other towards or away from the patient's mouth as in other illustrated arrangements herein. The device 100 is separable from the mask in some arrangements. In other arrangements, device 100 is not removable from the mask. For example, device 100 can be connected at the mask at the opening in the mask.

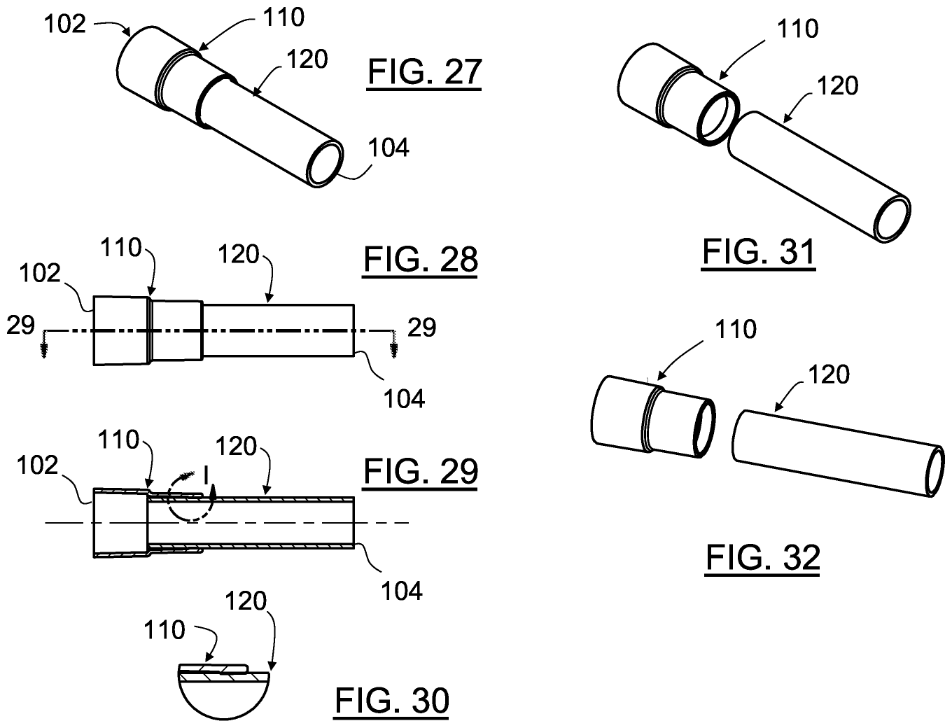

In one such arrangement, as shown in FIGS. 27-32, the device 100 can include separate pieces, outer tube 110 and inner tube 120 as shown, and the separate pieces are static relative to each other insofar as the pieces do not retract or extend into or out of the mask relative to each other. In this arrangement, outer tube 110 and inner tube 120 are separable. Inner tube 120 may be removably attached to outer tube 110 by inserting inner tube 120 into an end of outer tube 110. Inner tube 120 may be removably attached to outer tube 110 via a snap-fit mechanism. In the illustrated arrangement, outer tube 110 includes an internal abutment. The internal abutment may limit the depth to which inner tube 120 may be inserted into outer tube.

In other words, in this arrangement, the tubular body 100 comprises a first section 110 defining the first end 102 and a second section 120 defining the end portion 106 and distinct from the first section 110, and the first and second sections are removably interconnectable in fixed relation to each other. Thus, in an assembled or interconnected condition, the tubular body 100 has a prescribed fixed length for extending into the cavity 20 by a prescribed depth to present the end portion for gripping by the patient's lips.

According to another arrangement, outer tube 110 and inner tube 120 are integral with each other. For example, outer tube 110 and inner tube 120 are a single piece or mold and form a static device that is removable from the mask in some arrangements and not removable from a mask in other arrangements. In other words, in this arrangement, the conduit is formed from a unitary tube.

According to an arrangement, outer tube 110 and inner tube 120 may not extend or retract in relation to each other, and inner tube 120 is fixed or attached to outer tube 110 in an extended position. Inner tube 120 descends inside a respiratory mask toward a patient's lips. According to an arrangement, outer tube 110 and inner tube 120 are attached to the respiratory mask and not removable. According to an arrangement, outer tube 110 and inner tube 120 are removable from the respiratory mask.

According to an arrangement, ventilation and oxygenation accessory device 100 includes a respiratory mask. The respiratory mask may be disposed between an end of outer tube 110 proximate inner tube 120 and an end of inner tube 120 proximate outer tube 110. The respiratory mask may be disposed at a point along a length of outer tube 110. According to an arrangement, the respiratory mask is disposed at a point along a length of inner tube 120. The respiratory mask may be integral with outer tube 110 and inner tube 120. The respiratory mask may enable ventilation and pre-oxygenation accessory device 100 to be used with an oxygenation circuit more easily and more quickly by eliminating the need to interpose ventilation and oxygenation accessory device between a length of tubing connected to an oxygen source and a distinct respiratory mask. Outer tube 110, inner tube 120 and an aperture in the respiratory mask may define a continuous passage for oxygen delivered to ventilation and oxygenation accessory device 100 via a length of tubing connected at a first end to an oxygen source and at a second end to outer tube 110.

In all the illustrated arrangements, the end portion 106 of the tubular body is cylindrical in shape. That is, the end portion 106, which is grippable by the patient's lips, has uniform cross-sectional shape from the second end 104 to a spaced upstream location along the second section 120, which portion is receivable in the patient's mouth. More specifically, the outer surface of the end portion 106 is cylindrical in shape.

In all the illustrated arrangements, the tubular body 100 extends linearly between the first and second ends thereof 102, 104 in a working condition of the conduit in which the end portion 106 is arranged at the mouth of the patient so as to be available for gripping by the lips of the patient. When the conduit has adjustable length between the first and second ends 102, 104, the working condition corresponds to the extended condition, which in turn is associated with the extended position which is relative to the mask in the system.

Ventilation and oxygenation accessory device 100 may be constructed from materials which provide sufficient mechanical strength and durability to perform reliably over time and/or which are compatible with one or more methods of sterilization. According to an arrangement, pre-oxygenation accessory device 100 may be constructed of aluminum, stainless steel, silicone, thermoplastics, or a combination of suitable materials. Manufacturing costs, compatibility with one or more methods of sterilization, recyclability, weight, lifespan and mechanical strength may be considerations informing the choice of materials, construction technique and design choices of ventilation and oxygenation accessory device 100.

According to an arrangement, there is provided a method of improving the efficacy of a ventilation and/or an oxygenation procedure, comprising: inserting a first end of an outer tube into an end of a length of tubing connected to an oxygen or gas source; inserting a second end of an outer tube into an aperture in a respiratory mask; applying the respiratory mask to the face of a patient requiring ventilation and/or oxygenation; extending an inner tube, disposed within the outer tube, through the aperture in the respiratory mask such that the inner tube is proximate the patient's mouth; and instructing the patient to wrap their lips around an end of the inner tube. According to a further arrangement, the step of extending the inner tube comprises: rotating a toothed wheel mounted within an aperture in a wall of the outer tube, such that the toothed wheel engages a length of grooved track embedded in an outer face of the inner tube and running parallel to a length thereof. The step of extending the inner tube may comprise gripping a lever extending from a wall of the inner tube and through a primary channel in a wall of the outer tube; and sliding the lever toward an end of the primary channel. According to another arrangement, the step of extending the inner tube comprises retracting a compressive element disposed between the inner tube and the outer tube.

The extension of the inner tube through the aperture in the respiratory mask toward the patient's mouth enables the patient to independently engage the inner tube and, consequently, the oxygenation circuit. With the inner tube extended, oxygen supplied to the ventilation and oxygenation accessory device from an oxygen source is directed to the patient exclusively through the inner tube. By instructing the patient to wrap their lips around the inner tube, a medical practitioner ensures that all oxygen being delivered through the oxygenation circuit is directed into the patient's lungs. Where the patient is unconscious and, therefore, cannot engage the inner tube in the manner described, the inner tube may be retracted via an inner tube retraction and extension mechanism, such that the oxygen being delivered to the respiratory mask fills the entire mask.

According to an arrangement, there is provided a method of improving the efficacy of a ventilation and pre/or-oxygenation procedure, comprising: inserting a first end of an outer tube into an end of a length of tubing connected to a gas and/or oxygen source; inserting a first end of an inner tube into a second end of the outer tube; inserting the second end of the outer tube into an aperture in a respiratory mask; applying the respiratory mask to the face of a patient requiring ventilation and/or oxygenation; and instructing the patient to wrap their lips around a second end of the inner tube.

In at least one arrangement, ventilation and oxygenation accessory device 100 is inserted at a mask. That is, the conduit connects within the system 10 in series between the supply tube 14 and the mask 17.

In at least one arrangement, ventilation and oxygenation accessory device 100 extends into and retracts from a cavity defined by a mask, as shown in FIGS. 3A and 3B.

As previously mentioned, in at least one arrangement, ventilation and oxygenation accessory device 100 is designed to be incorporated into the ventilation and/or oxygenation circuit between a gas conduit and the respiratory mask. Ventilation and oxygenation accessory device 100 includes an inner tube 120 and an outer tube 110, wherein the inner tube 120 may be retracted such that ventilation and oxygenation accessory device 100 may be permanently incorporated into the ventilation and/or oxygenation circuit and used only when appropriate. The inner tube 120 can retract fully or partially into the outer tube 110 for the mask circuit to be used as an oxygenation circuit prior to intubation. According to an arrangement, the outer tube 110 may include a fuse to create a seal with the inner tube 120, and the inner tube 120 may include a rubber seal that attaches to the outer tube 110. In the retracted position, the inner tube 120 is fully or partially retracted within the space within the outer tube 110. In the extended position, the inner tube 120 extends into the cavity defined by the respiratory mask toward a patient's mouth. In use, a patient engages the extended inner tube 120 to create a seal between their lips and the inner tube 120 during ventilation and/or oxygenation. According to an arrangement, ventilation and oxygenation accessory device 100 provides increased integrity of the seal between the patient and the oxygen mask to avoid leaks during the oxygenation process. The method of extending and retracting the inner tube 120 may use corrugated tubing (which may operate similarly to a spring mechanism) or may comprise a gear and track mechanism wherein a spinning gear incorporated in a wall of the outer tube engages a track disposed on an outer surface of the inner tube. According to arrangements, inner tube 110 and outer tube 120 can be moved in relation to each other and/or extended into and retracted from a mask using various mechanisms. Dimensions may differ from that shown.

This provides an arrangement of oral delivery conduit for bridging, within an oronasal cavity formed by the mask, a distance between an inlet of the oronasal cup, proximate to which the downstream end of the supply tube is located, and the patient's mouth, so that the ventilation gas is released at a position closer to the patient's mouth for inhaling by the patient using the mouth. Especially when the patient wraps their lips around the end portion of the conduit, a transfer of ventilation gas to a respiratory system of the patient, and particularly their lungs, is enhanced as compared to flooding the oronasal cavity with the ventilation gas because the patient's lips can substantially conform to an outer surface of the body of the conduit to form a relatively impermeable seal therebetween as compared to conformability of the mask to the patient's face for sealing purposes. In this manner, for a prescribed duration of time, a greater proportion of the supplied ventilation gas may be delivered to the patient's lungs, and accordingly a lesser proportion of the supplied ventilation gas may be lost to a surrounding environment.

There is also disclosed herein a method for artificially ventilating a patient comprising the steps of:
providing a source of ventilation gas 12 for delivery to the patient;
providing a supply tube 14 operatively connected to the source 12 to convey the ventilation gas therefrom and to a release location in proximal spaced relation to a face F of the patient 1 and defined by an end 15B of the supply tube distal to the source 12;
providing a mask 17 in operative association with the supply tube to form at the release location a cavity 20 around a mouth M and nose N of the patient for containing the ventilation gas relative to the face of the patient;
providing a tubular conduit 100 distinct from the supply tube 14 and fluidically communicated with the distal end thereof 15B to convey the ventilation gas from the supply tube 15 and towards the mouth M of the patient;
locating the mask 17 on the patient's face F to form the cavity 20; and
locating the tubular conduit 100 in the cavity 20 to present an end portion thereof 106, which defines a free end 104 of the tubular conduit, for gripping by lips of the patient for oral delivery of the ventilation gas.

In some arrangements, the mask 17 is operatively associated with the supply tube 14 by the tubular conduit 100 which mechanically interconnects the tube 14 and the mask 17. However, downstream of the supply tube the ventilation gas is guided by the conduit 100 towards the patient's mouth, and not the cup 18.

In some arrangements, locating the tubular conduit in the cavity comprises extending the end portion 106 towards the mouth M of the patient from a retracted position in which the free end 104 is in outwardly spaced relation from the mouth M of the patient such that the end portion 106 is unavailable for gripping by the lips of the patient. That is, in the retracted position, the free end 104 is spaced further outwardly from the mouth M than in an extended position thereof, in which the end portion 106 is grippable by the patient's lips.

The method may further include, after locating the tubular conduit 100 in the cavity 20, substantially removing the tubular conduit 100 from the cavity and maintaining the mask on the patient's face F so that the mask acts to convey the ventilation gas for oronasal delivery to the patient, as shown for example in FIG. 3B. This may be performed when the patient loses consciousness and is unable to grip the conduit.

In one such arrangement, substantially removing the tubular conduit from the cavity comprises retracting the end portion 106 away from the mouth of the patient from an extended position in which the end portion 106 is at the mouth of the patient to be available for gripping by the lips of the patient, as shown for example in FIG. 3A.

Preferably, retracting the end portion 106 is performed while the mask 17 is on the patient's face forming the cavity. As such, the ventilation gas can continue to act on the patient while the conduit is retracted away from the patient's mouth.

The scope of the claims should not be limited by the preferred embodiments set forth in the examples but should be given the broadest interpretation consistent with the specification as a whole.

1. A system for artificially ventilating a patient comprising:
a source of ventilation gas for delivery to the patient;
a supply tube configured to convey the ventilation gas and having an upstream end in fluidic communication with the source to receive the ventilation gas therefrom and a downstream end configured to release the gas;
a mask comprising an oronasal cup configured to form a cavity around a mouth and nose of the patient and an inlet in the oronasal cup and operatively supported at the downstream end of the supply tube;
an oral delivery conduit in the form of a tubular body configured to be received in the cavity and defining a path for flow of the ventilation gas;
wherein the tubular body has a first end in fluidic communication with the downstream end of the supply tube and extends therefrom into the cavity and to a second end arranged in proximal relation to the mouth of the patient, so as to convey the ventilation gas from the supply tube and towards the patient's mouth;
wherein an end portion of the tubular body defining the second end thereof is arranged for sealingly gripping by lips of the patient for oral delivery of the ventilation gas, such that the mask forms a secondary seal for containing the ventilation gas relative to a face of the patient.

2. The system of claim 1 wherein the end portion of the tubular body is cylindrical in shape.

3. The system of claim 1 wherein, in a working condition in which the end portion is arranged at the mouth of the patient so as to be available for gripping by the lips of the patient, the tubular body extends linearly between the first and second ends thereof.

4. The system of claim 1 wherein the tubular body comprises an outer section defining the first end and an inner section defining the end portion and operatively interconnected to the outer section to provide a continuous path for flow of the ventilation gas, and wherein the outer section is arranged fluidically in series between the supply tube and the mask.

5. The system of claim 1 wherein, when the oronasal cup has a rim configured for sealing engagement with the patient's face, the tubular body has adjustable length between the first and second ends so as to be configurable between a retracted position in which the second end is arranged closer to the inlet of the mask than to the rim of the oronasal cup and an extended position in which the second end is arranged closer to the rim of the oronasal cup than to the inlet.

6. The system of claim 5 wherein, when the tubular body comprises a first section defining the first end and a second section defining the end portion and configured for movement relative to the first section to provide the adjustable length, the first and second sections are operatively interconnected for relative movement by a mechanism comprising a track supported on one of the first and second sections and a wheel operatively supported on another one of the first and second sections and matably engaged with the track.

7. The system of claim 5 wherein, when the tubular body comprises a first section defining the first end and a second section defining the end portion and configured for movement relative to the first section to provide the adjustable length, the second section is fixedly attached to the first section and comprises a flexible length of tubing deformable crosswise to a longitudinal direction of the tubular body for varying the adjustable length.

8. The system of claim 7 wherein, the first section forms an outer housing having an interior through which the second section extends, and the tubular body includes a deflector supported in the outer housing and movable relative thereto in the crosswise direction for deformatively engaging the second section.

9. The system of claim 5 wherein, when the tubular body comprises a first section defining the first end and a second section defining the end portion and configured for movement relative to the first section to provide the adjustable length, the first section forms an outer housing having an interior through which the second section extends, and the second section is fixedly attached to the first section and is compressible in a longitudinal direction of the tubular body for providing the adjustable length.

10. The system of claim 5 wherein, when the tubular body comprises a first section defining the first end and a second section defining the end portion and configured for movement relative to the first section to provide the adjustable length, the first and second sections are telescopically slidably interconnected and are rotatable to each other around a longitudinal axis of the tubular body, and wherein an outer one of the first and second sections includes a slot extending longitudinally of the tubular body and an inner one of the first and second sections includes a catch received in the slot, wherein the slot includes jogs in crosswise directions to the longitudinal direction for maintaining the first and sections at a selected length.

11. The system of claim 1 wherein the tubular body comprises a first section defining the first end and a second section defining the end portion and distinct from the first section, and wherein the first and second sections are removably interconnectable in fixed relation to each other.

12. A device for use in a system to artificially ventilate a patient, wherein the system includes a source of ventilation gas for delivery to the patient, a supply tube configured to convey the ventilation gas and having an upstream end in fluidic communication with the source to receive the ventilation gas therefrom and a downstream end configured to release the gas, and a mask comprising an oronasal cup configured to form a cavity around a mouth and nose of the patient and an inlet in the oronasal cup and operatively supported at the downstream end of the supply tube, the device comprising:
a tubular body defining a path for flow of the ventilation gas;
wherein the tubular body is configured to be received in the cavity;
wherein the tubular body has a first end arranged in fluidic communication with the downstream end of the supply tube and a second end arranged in proximal relation to the mouth of the patient, so as to be arranged to extend from the downstream end of the supply tube and into the cavity towards the patient's mouth;
wherein an end portion of the tubular body defining the second end thereof is arranged for sealingly gripping by lips of the patient for oral delivery of the ventilation gas, such that the mask forms a secondary seal for containing the ventilation gas relative to a face of the patient.

13. A method for artificially ventilating a patient comprising:
providing a source of ventilation gas for delivery to the patient;
providing a supply tube operatively connected to the source to convey the ventilation gas therefrom and to a release location in proximal spaced relation to a face of the patient and defined by an end of the supply tube distal to the source;
providing a mask in operative association with the supply tube to form at the release location a cavity around a mouth and nose of the patient for containing the ventilation gas relative to a face of the patient;
providing a tubular conduit distinct from the supply tube and fluidically communicated with the distal end thereof to convey the ventilation gas from the supply tube and towards the mouth of the patient;
locating the mask on the patient's face to form the cavity; and
locating the tubular conduit in the cavity to present an end portion thereof, which defines a free end of the tubular conduit, for gripping by lips of the patient for oral delivery of the ventilation gas.

14. The method of claim 13 wherein locating the tubular conduit in the cavity comprises extending the end portion towards the mouth of the patient from a retracted position in which the free end is in outwardly spaced relation from the mouth of the patient such that the end portion is unavailable for gripping by the lips of the patient.

15. The method of claim 13 further including, after locating the tubular conduit in the cavity, substantially removing the tubular conduit from the cavity and maintaining the mask on the patient's face so that the mask acts to convey the ventilation gas for oronasal delivery to the patient.

16. The method of claim 15 wherein substantially removing the tubular conduit from the cavity comprises retracting the end portion away from the mouth of the patient from an extended position in which the end portion is at the mouth of the patient to be available for gripping by the lips of the patient.

17. The method of claim 16 wherein retracting the end portion is performed while the mask is on the patient's face forming the cavity.
